(19) United States
(12) Patent Application Publication
(10) Pub. No.: US 2005/0070931 A1
(43) Pub. Date: Mar. 31, 2005

(54) Method and apparatus for creating a restriction in the stomach or other anatomical structure

(76) Inventors: Rhodemann Li, Darien, CT (US); Lehmann K. Li, Milford, CT (US)

Correspondence Address: Mark J. Pandiscio;Pandiscio & Pandiscio, P.C., 470 Totten Pond Road, Waltham, MA, 02451-1914, US

(21) Appl. No.: 10/913,098

(22) Filed: Aug. 6, 2004

Related U.S. Application Data
(60) Provisional application No. 60/492,562, filed on Aug. 6, 2003.
(60) Provisional application No. 60/523,829, filed on Nov. 20, 2003.

(51) Int. Cl. A61B 17/08

(52) U.S. Cl. 606/151

(57) ABSTRACT

A method is for restricting food volume capacity of the stomach. The method comprises manipulating tissue from within the stomach to create an upper pouch and a lower pouch, and the tissue between the upper pouch and the lower pouch forming an opening therethrough, the upper pouch configured adjacent the gastroesophageal junction, the lower pouch configured adjacent the pylorus, and the opening having a periphery formed by drawing together the tissue. A device for creating a restriction in the stomach. The device comprises a retractor configured for deployment through the esophagus into the stomach; a plurality of arms selectively deployable from the retractor, each of the plurality of arms configured to position a securement device adjacent to a given location of tissue of the stomach from within the stomach; and deployment means for deploying the securement device positioned by each of the plurality of arms to the given location of the tissue of the stomach from within the stomach.

### REFERENCE TO PENDING PRIOR PATENT APPLICATIONS

This patent application claims benefit of:
(1) pending prior U.S. Provisional Patent Application Ser. No. 60/492,562, filed Aug. 6, 2003 by Rhodemann Li et al. for IMPLANTING DEVICES IN BIOLOGICAL TISSUE (Attorney's Docket No. ENDO-01 PROV); and
(2) pending prior U.S. Provisional Patent Application Ser. No. 60/523,829, filed Nov. 20, 2003 by Rhodemann Li et al. for IMPLANTING DEVICES IN BIOLOGICAL TISSUE (Attorney's Docket No. ENDO-02 PROV).

The two above-identified patent applications are hereby incorporated herein by reference.

### FIELD OF THE INVENTION

This invention is related to methods and systems for implanting devices in biological tissue in general, and more particularly to methods and systems for treating medical diseases or conditions of the gastrointestinal system and other anatomical systems. Furthermore, the invention is directed to the use of devices as temporary or permanent implants in biological tissue.

### BACKGROUND OF THE INVENTION

Obesity is a worldwide public health crisis. Obesity is a disease with serious morbidity and mortality implications for sufferers. Based on the 1999-2000 National Health and Nutrition Examination Survey published by the National Center for Health Statistics, approximately 59 million U.S. adults (31%) are obese, of which 11 million are severely obese. Approximately 325,000 U.S. adults die of causes attributable to obesity each year. The worldwide incidence of obesity is about 250 million people, with the prevalence also increasing rapidly in numerous developing nations worldwide. The implications of obesity on healthcare resources are enormous as obesity is a known risk factor for many diseases and conditions including diabetes, heart disease, stroke, hypertension, osteoarthritis and some forms of cancer.

### SUMMARY OF THE INVENTION

Accordingly, one object of the present invention is to provide a method and apparatus to treat diseases or medical conditions such as, but not limited to, obesity in a minimally invasive manner. In particular, endoscopic techniques are far less invasive than surgical approaches currently used in gastric bypass and laparoscopic band procedures.

Another object of the present invention is to provide an endoscopic method and apparatus to deliver or implant devices to affect a biologic process such as, but not limited to, peristalsis, satiety or the digestive process.

Yet another object of the present invention is to provide an endoscopic method for restricting food volume capacity of the stomach.

Another object of the present invention is to provide an endoscopic device for restricting food volume of the stomach.

A further object of the present invention is to provide an endoscopic method and apparatus for creating an upper pouch, a lower pouch and a stoma within the stomach to restrict food volume of the stomach.

A still further object of the present invention is to provide an endoscopic method and apparatus for restricting food volume of the stomach in which secured elements are positioned in tissue at given locations within the stomach.

With the above and other objects in view, as will hereinafter appear, there is provided a method for restricting food volume capacity of the stomach, the method comprising:
manipulating tissue of an interior wall of the stomach from within the stomach so as to create an upper pouch, a lower pouch and an opening connecting the upper pouch and the lower pouch, the upper pouch being located adjacent the gastroesophageal junction, the lower pouch being located adjacent the pylorus, and the opening having a periphery formed by drawing together the tissue of the interior wall of the stomach.

In accordance with a further feature of the present invention, there is provided a method for restricting food volume capacity of the stomach, the method comprising:
placing a plurality of elements for gripping the tissue within the stomach at given locations;
drawing the plurality of elements for gripping the tissue toward one another so as to draw the given locations of the stomach toward one another; and
fixing the position of the drawn-together locations of the stomach relative to one another;
whereby to divide the stomach into an upper pouch and a lower pouch, with an opening connecting the upper pouch to the lower pouch.

In accordance with another feature of the present invention, there is provided a method for restricting food volume capacity of the stomach, the method comprising:
drawing toward one another given portions of tissue of an interior wall of the stomach from within the stomach; and
fixing the position of the given portions of the stomach drawn toward one another;
whereby to divide the stomach into an upper chamber and a lower chamber, with an opening connecting the upper chamber to the lower chamber.

In accordance with a further feature of the present invention, there is provided a device for creating a restriction in the stomach, the device comprising:
a retractor configured for deployment through the esophagus into the stomach;
a plurality of arms selectively deployable from the retractor, each of the plurality of arms configured to position a securement device adjacent to a given location of tissue of the stomach from within the stomach; and
a deployment mechanism for deploying the securement device positioned by each of the plurality of arms to the given location of the tissue of the stomach from within the stomach.

In accordance with another feature of the present invention, there is provided a device for creating a restriction in the stomach, the device comprising:
a track configured for deployment through the esophagus into the stomach;
a securement device mounted to the track, such that the securement device is positioned adjacent to a given location of tissue of the stomach from within the stomach; and
a deployment mechanism for deploying the securement device to the given location of tissue of the stomach from within the stomach.

The above and other features of the invention, including various novel details of construction and combinations of parts and method steps, will now be more particularly described with reference to the accompanying drawings and pointed out in the claims. It will be understood that the particular devices and method steps embodying the invention are shown by way of illustration only and not as limitations of the invention. The principles and features of this invention may be employed in various and numerous embodiments without departing from the scope of the invention.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIGS. 1A and 1B are schematic illustrations of markings on the inside wall of the stomach indicating preferred attachment locations to create an upper pouch, a lower pouch, and a stoma between the pouches;

FIGS. 1C-1F are schematic cross-sectional illustrations showing the creation of a stoma in the stomach in accordance with a preferred embodiment of the present invention;

FIG. 1G is a schematic illustration showing a stoma from the lower pouch into the upper pouch;

FIG. 1H is a schematic exterior illustration showing a pouch created in the stomach accordance with a preferred embodiment of the present invention;

FIG. 2 is a diagrammatic perspective view showing an endoscopic retractor instrument for creating a gastric pouch and stoma in accordance with a preferred embodiment of the present invention;

FIG. 3 is a schematic view showing attachment elements for creating a stoma in accordance with a preferred embodiment of the present invention;

FIGS. 4-6 are schematic views showing a retractor arm for placing an attachment element shown in FIG. 3;

FIG. 7 is a schematic endoscopic view showing normal gastric rugal folds of a stomach;

FIG. 8 is a schematic endoscopic view showing the inner wall of the stomach inflated to smooth the rugal folds shown FIG. 7;

FIGS. 9 and 10 are schematic views showing a scaffold device for creating a stoma in accordance with the present invention;

FIGS. 11-19 are schematic views showing an endoscopic instrument configured to place attachment elements within the stomach in accordance with the present invention;

FIGS. 20-24 are schematic illustrations showing fixation of placations within a stomach for creating a stoma in accordance with the present invention;

FIGS. 25-31 are schematic drawings showing a tissue attachment instrument and a suture path created by the instrument for forming a stoma in accordance with the present invention; and

FIGS. 32-36 are schematic illustrations showing fixation of an adjustable ring in the stomach wall in accordance with a preferred embodiment of the present invention.

### DETAILED DESCRIPTION OF THE PREFERRED EMBODIMENTS

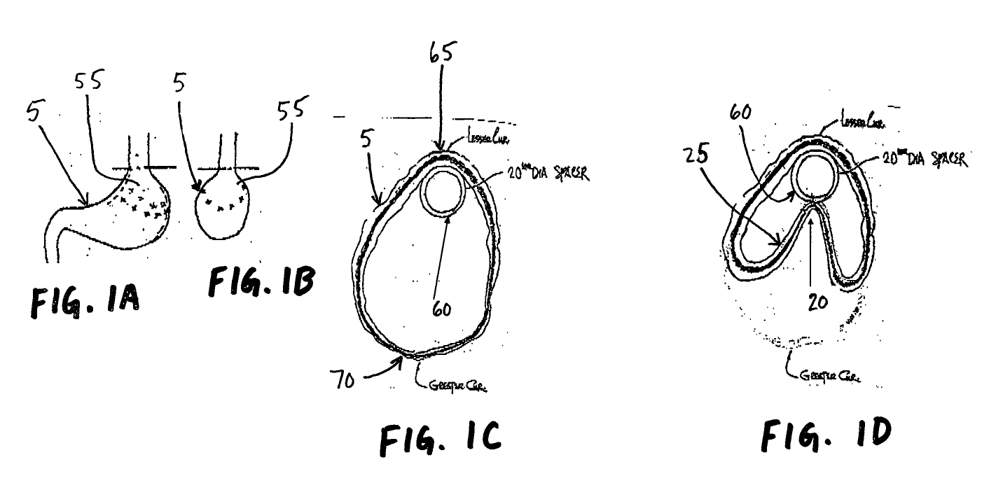

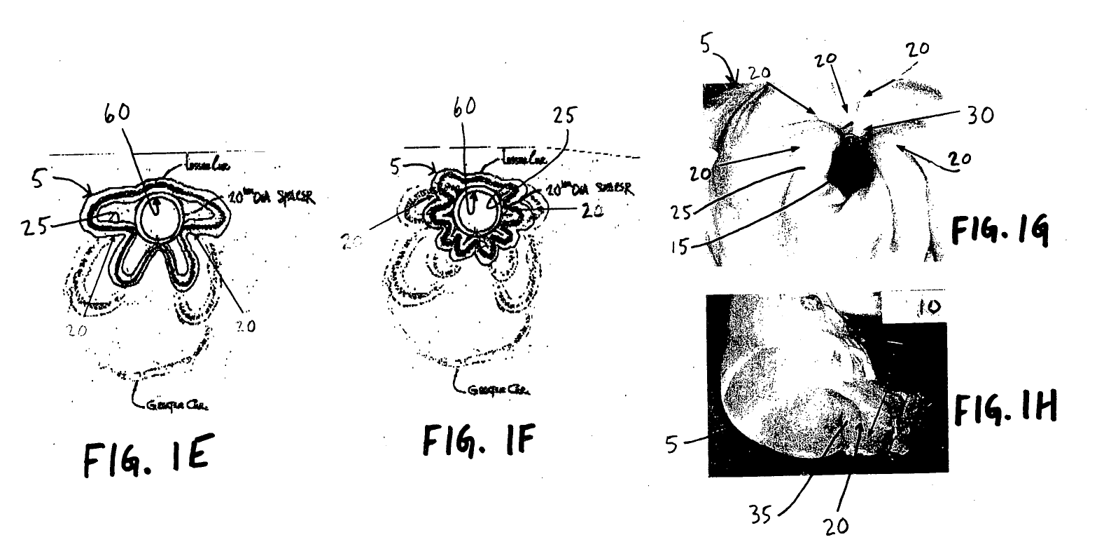

Looking at FIGS. 1A-1H, there is shown one preferred form of an endoscopic procedure to restrict the food volume capacity of the stomach 5 by creating a small gastric pouch 10 and stoma 15. This procedure is preferably accomplished by circumferentially, or nearly circumferentially, attaching at least two inward or outward folds, tents or plications 20 of tissue 25 in a portion of the stomach 5. For example, this attachment may be effected using attachment elements 30, such as sutures, staples, clips, barbs, hooks, welding, gluing, or any combination thereof, etc. Alternatively, the circumferential, or nearly circumferential attachment of a portion of the stomach may involve at least one anchor point in the stomach tissue which is not folded or plicated.

Pouch 10 is preferably similar to the type of pouch created during a gastric band or bypass surgical procedure, i.e., a small size pouch located in the upper portion of the stomach adjacent to the gastroesophageal junction.

Stoma 15 is created at the lower (or distal) end 35 of pouch 10 by apposing and attaching a series of inwardly or outwardly folded portions 20 of tissue 25, either in an interrupted or continuous fashion. Folds 20 may be horizontal, vertical or any other orientation. Various means to adjust the diameter of stoma 15 may be used, such as by repeatedly drawing together and securing more than one folded portion 20 of tissue 25 over a prior fold 20, or by folding and securing an adjacent portion 20 of tissue 25. Thus, by way of example but not limitation, FIGS. 1C-1F show how various locations on the inner wall of stomach 5 may be drawn together so as to create the desired stoma 15. Significantly, with the present invention, stoma 15 may be created using apparatus located within the interior of stomach 5, i.e., the pouch 10 and stoma 15 may be created endoluminally.

In order to encourage healing of the apposed tissue folds 20, certain layers of the stomach 5, for example, the mucosa or the submucosa, may be denuded or stripped away prior to securement. The stripping away of the mucosa or submucosa may be accomplished in any number of ways, which include, but are not limited to, electrocautery and RF ablation, and may be performed using a component of the endoscopic instrument or a secondary instrument. By denuding certain layers of tissue 25, once healing has occurred the attachment sutures, staples, glue or other attachment devices become redundant, and thereby the strength and longevity of stoma 15 becomes a function of tissue viability. In a preferred embodiment of the present invention, bolsters or tissue ingrowth mesh material may be added to the tissue folds so as to augment healing.

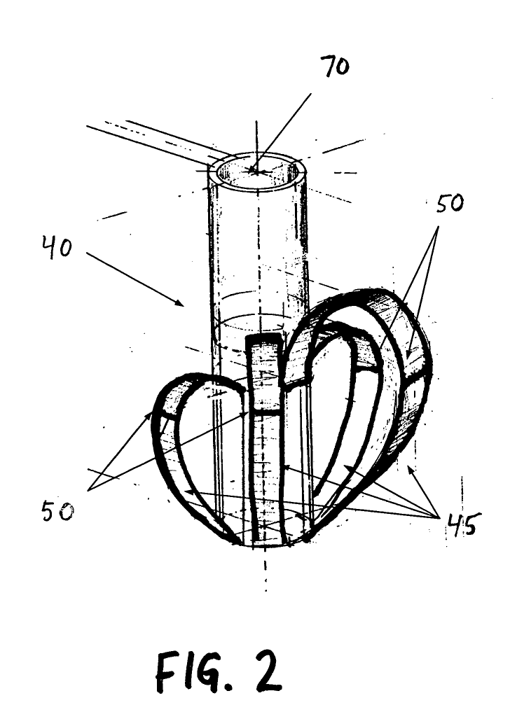

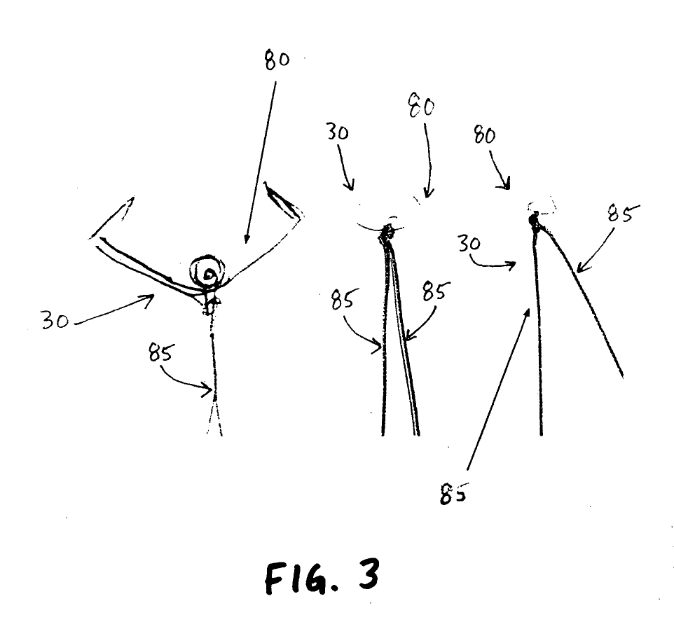

Referring to FIG. 2, in a preferred form of the present invention, there is provided an endoscopic retractor 40 which may be used to create a small gastric pouch 10 and stoma 15 in a repeatable and consistent manner. This is preferably accomplished by providing a pouch sizing component 45 and accurate tissue securement markings 50 on an endoscopic instrument 40. One means of sizing pouch 10 with instrument 40 is to have markings 50 at one or more specific locations on at least one flexible distal instrument portion 45 so as to indicate the locations at which the attachment elements 30 (for example, sutures, staples, or glue) are to be secured onto biological tissue 25. In another, more preferred manner of sizing pouch 10, the attachment element 30 is preferably housed within an integrated part of the flexible distal instrument portion 45, i.e., at the markings 50. Since the attachment element 30 is placed accurately at specific locations onto tissue 25 due to its fixed location on the flexible distal instrument portion 45 during deployment, pouch 10 is created with a predetermined volume. Once adjacent attachment elements 30 are secured to each other so as to create the desired tissue folds, stoma 15 is created with the predetermined small gastric pouch 10.

In one preferred embodiment of the present invention, short-term excess weight loss is attained by using absorbable sutures 30 to create the small gastric pouch 10 and stoma 15. Once the absorbable sutures have lost their structural integrity, and especially if the mucosa and submucosa have been left intact so as to minimize any tissue-to-tissue regrowth, stoma 15 increases in diameter such that the small gastric pouch 10 and stoma 15 no longer serve to significantly restrict the volume of food a patient can consume before feeling satiety.

Alternatively, the use of absorbable or biodegradable attachment elements 30 including, for example, clips, staples, or glue, are placed into the tissue to affect short term excess weight loss. Short-term excess weight loss is also attained by removing or cutting the attachment elements 30 at some time period after the initial procedure so as to release or loosen the small gastric pouch 10 and stoma 15.

In an alternative embodiment of the present invention, there is provided a method for creating a stoma (not shown) to augment sphincters or valves in other minimally invasive procedures such as those used to treat incontinence, gastroesophageal reflux, cervical cerclage, or heart valves.

In accordance with the present invention as disclosed herein, and referring again to FIGS. 1A and 1B, there is shown a front view (FIG. 1B) and side view (FIG. 1A) of stomach 5 with markings “x” to indicate where tissue folds 20 (FIGS. 1C-1H) are created, spaced nearly circumferentially encompassing both the greater and lesser curvatures of the stomach.

FIG. 1C shows a top cross-sectional view of the stomach 5 with a stoma creation tool 60 placed endoscopically adjacent to the lesser curvature 65 of stomach 5. Stoma creation tool 60 may be a simple knot rundown tool, or a parachute knot rundown ring, suture crimping tool or any other means to create stoma 15. Or stoma creation tool 60 may be the endoscopic retractor 40 discussed above. In an alternative preferred embodiment, stoma creation tool 60 is omitted and attachment elements 30 are attached directly to one another to create stoma 15.

Referring to FIG. 1D, there is shown a first tissue plication 20 created by pulling inward stomach wall 25 at the greater curvature 70 of stomach 5. FIGS. 1E-1F illustrate additional tissue folds 20 being pulled inward toward stoma creation tool 60.

Referring to FIG. 1G, there is shown (in a view from below) a stoma 15 created in a pig stomach specimen by knotting various sutures 30. FIG. 1G also illustrates various tissue folds 20 created by securing the attachment devices 30 (e.g., sutures) to each other. Denuding the mucosa or submucosa between the various tissue folds 20 brings blood supply between the apposed and secured tissue folds 20 and encourages healing to provide long-term strength to stoma 15. By creating tissue folds 20 generally circumferentially and securing folds 20 at various points, there is created a natural stoma 15. Referring to FIG. 1H, there is shown an exterior view of a pouch 10 created as discussed herein.

Thus, in one preferred form of the invention, the inner wall of the stomach is pulled inward at a given location so as to create a fold of tissue; this process is repeated at a plurality of locations about the interior of the stomach; and the plurality of folds are gathered together at their inner ends and joined to one another so as to collectively form the perimeter of an opening, which comprises the stoma.

And in one preferred form of the invention, each individual fold is created by anchoring a suture in the wall of the stomach and pulling that suture inwardly, and then the individual folds are joined to one another by tying the sutures together so as to form the perimeter of the stoma. Alternatively, each individual fold may be created by gripping the wall of the stomach (e.g., with suction) and then drawing it inward; adjacent folds may then be secured together (e.g., with staples, glue, etc.) so as to form the perimeter of the stoma.

And in one preferred form of the invention, the stomach tissue is debrided where the adjacent folds come together so as to facilitate tissue-to-tissue regrowth.

Referring again to FIG. 2, a perspective view of one preferred embodiment of the present invention illustrates the distal end of endoscopic retractor 40. Retractor 40 has an interior lumen 70 through which an endoscope (not shown) is preferably inserted and used to provide visualization of the procedure from within the stomach. Retractor 40 has at least one deployable, flexible arm 45. Located on each arm 45 is a defined position 50 for a securement device (not shown) and means of deploying the securement device (also not shown).

Referring now to FIG. 3, there is shown a securement device 30, such as a partially deployed staple 80 with a suture 85 pre-tied onto staple 80.

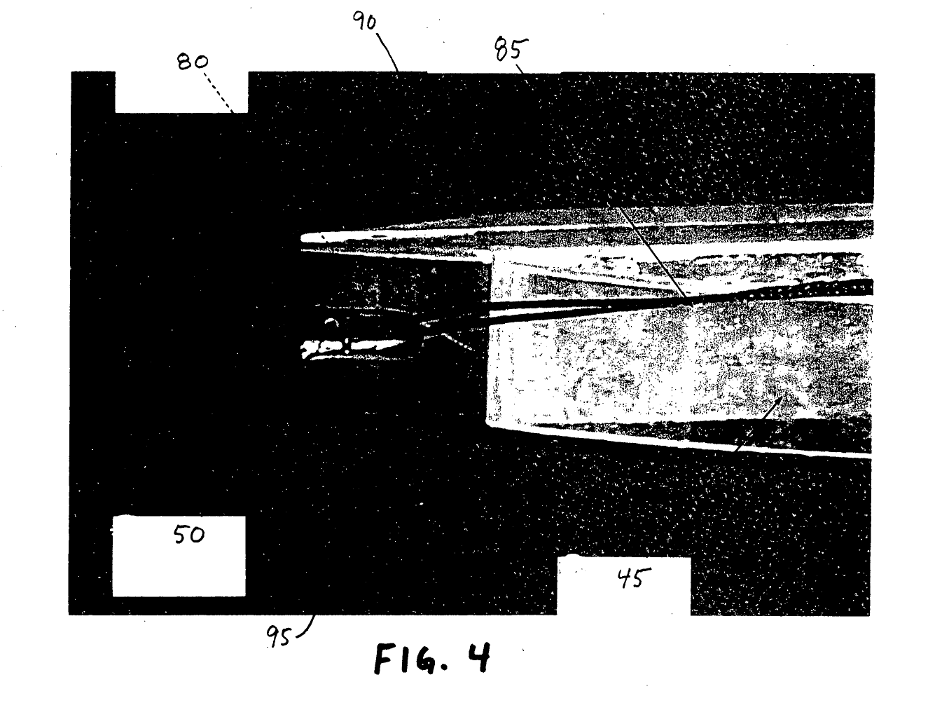

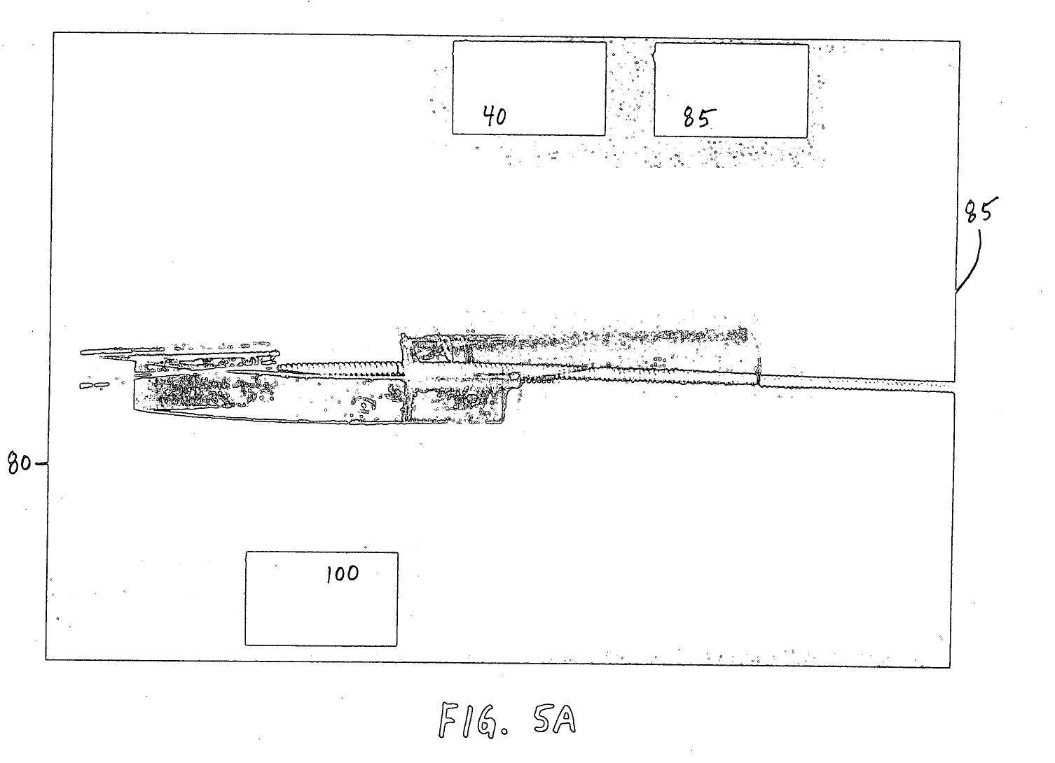

Referring now to FIG. 4, there is shown a close up view of a tool 90 which is adapted to deploy staple 80 which is stored within the distal end 95 of deployment tool 90.

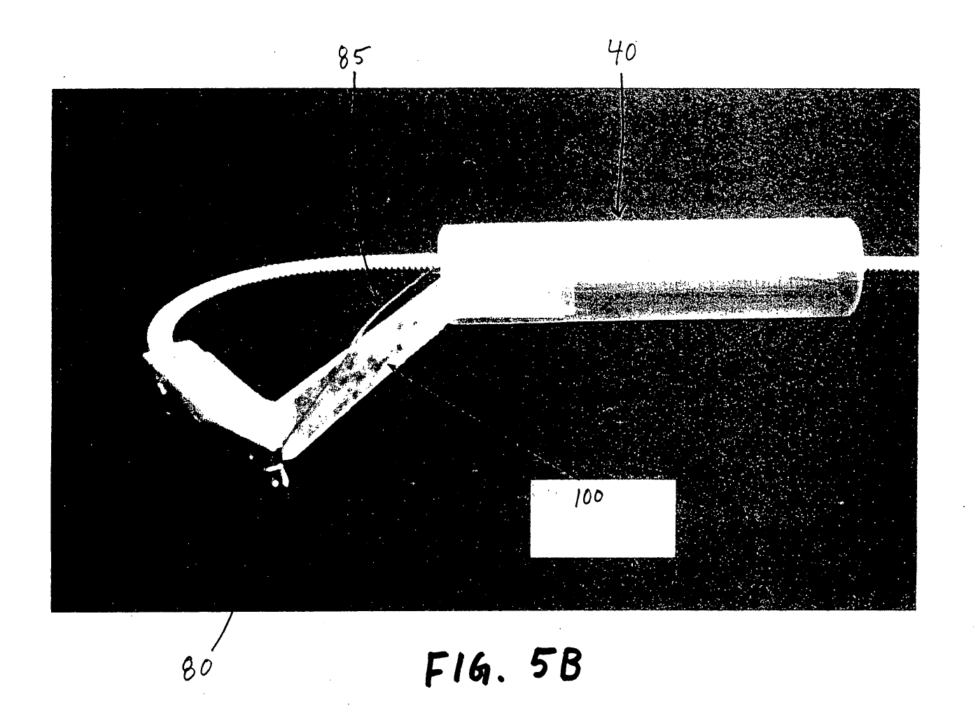

Looking at FIG. 5A, there is shown a retractor arm 100 used to control the placement of a staple 80 into tissue 25 (FIGS. 1C-1G). Pre-tied suture 85, attached to staple 80, is also shown.

A portion of retractor 40 is also shown in FIG. 5A, along with the deployable, flexible arm 100 and pre-tied suture 85 contained within the lumen of retractor 40. Looking at FIG. 5B, there is shown flexing of retractor arm 100 for positioning onto the tissue 25 (FIGS. 1C-1G) such that staple 80 is positioned and deployed into the tissue 25 (FIGS. 1C-1G) as desired.

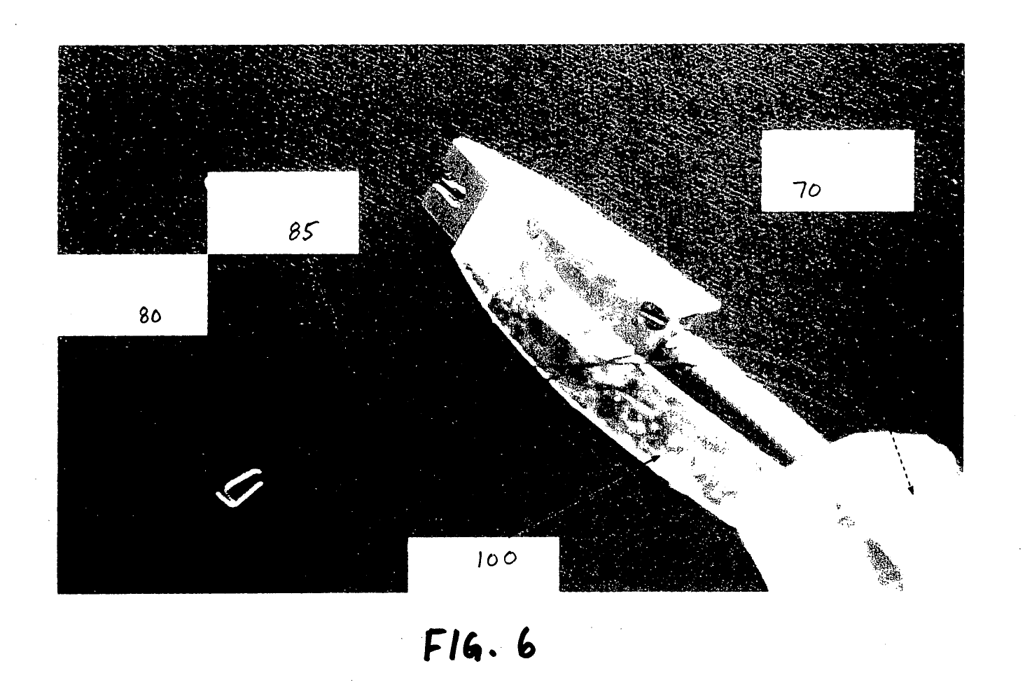

Looking at FIG. 6, there is shown a full deployment of staple 80 into tissue 25 (FIGS. 1C-1G), with the pre-tied suture 85 still contained within lumen 70 of retractor body 40. The deployment end 95 of arm 100 has been withdrawn from staple 80 and the arm 100 has been straightened to permit removal of retractor 40.

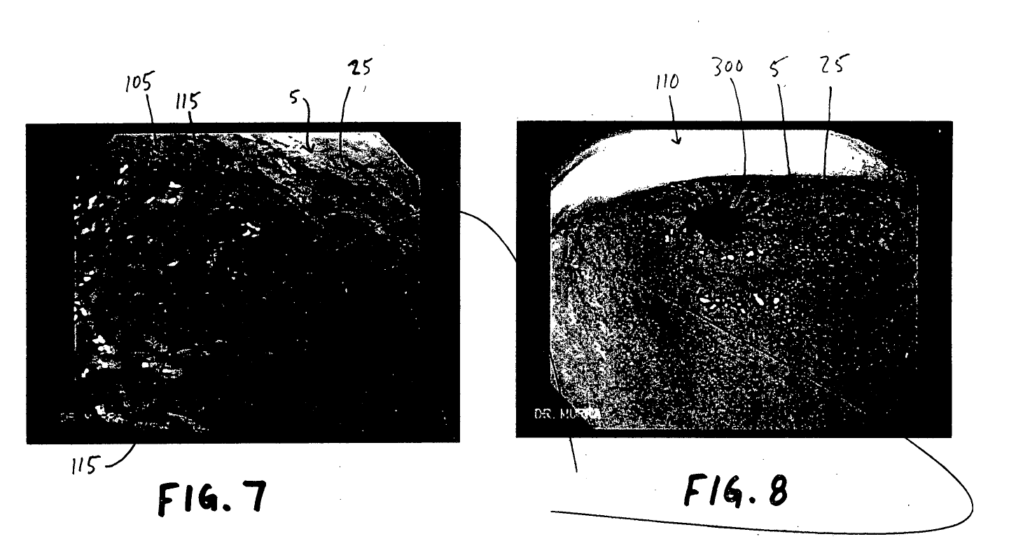

Referring to FIGS. 7 and 8, there is shown a natural stomach wall 105 (FIG. 7) and a stretched stomach wall 110 (FIG. 8). When taking an endoscopic approach, a preferred method for precise placement of the securement elements 30 (e.g., sutures, staples, glue, etc.) to create plications 20 and stoma 15 is to inflate stomach 5 with air or any biocompatible gas, fluid, device, etc., so as to stretch the stomach tissue 25, especially the rugal folds 115 (see FIG. 7). In this respect it should be appreciated that if the interior stomach wall 25 is not distended prior to placing securement element 30, the variability in thickness of the mucosa rugal folds 115 may affect the integrity and position of plications 20, and hence the integrity and dimensions of stoma 15.

To minimize variability, maximize healing and increase the accuracy of creating plications 20, alternative methods to inflating stomach 5 include the use of devices that expand or stretch the interior stomach wall 25 prior to placing securement elements 30.

One approach is to endoscopically deliver an instrument that can expand within the body of the stomach in such a way as to stretch interior wall 25 of stomach 5, thus smoothing out the rugal folds prior to placing the securement elements. Preferably, an instrument is delivered endoscopically in a collapsed form and then expanded once inside stomach 5 to effectively stretch interior stomach wall 25.

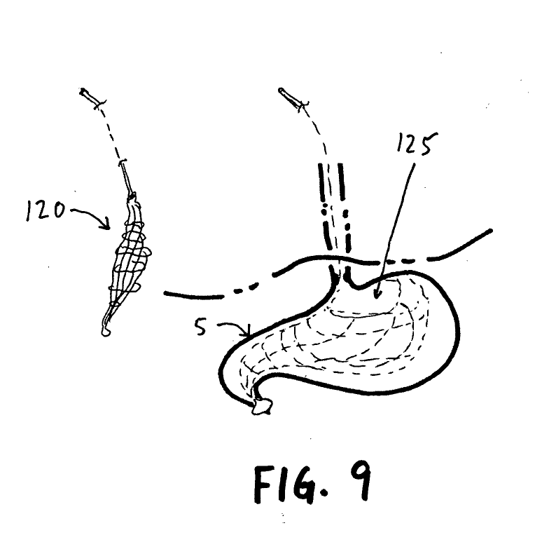

Referring now to FIGS. 9 and 10, there is shown scaffold device 120 which is inflatable, expandable, hinged, or has other mechanisms of increasing its dimensions, and is collapsible for easy removal. Scaffold device 120 is preferably configured as a balloon (FIG. 9) or a frame (FIG. 10). The area of expansion is preferably limited to areas of stomach 5 which are distinct from the intended areas where the securement elements 30 will be placed. There is shown an area 125 above scaffold device 120, and within stomach 5, which is not covered by scaffold device 120. Area 125 is suitable for placement of the securement elements 30. Preferably, securing elements 30 are delivered by one or more delivery devices positioned on scaffold device 120 adjacent to the periphery of area 125.

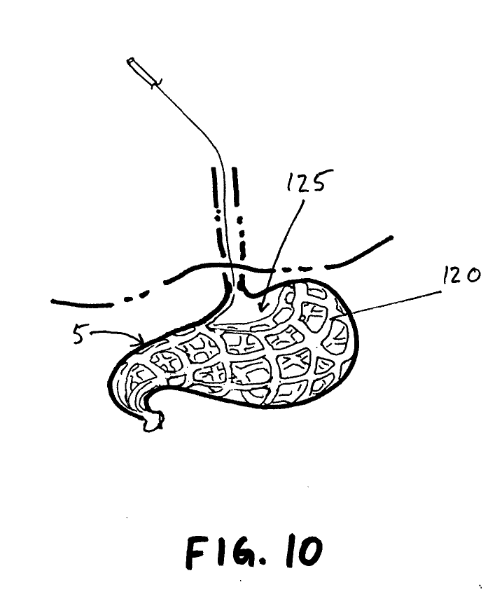

Referring now to FIGS. 11-19, there is shown an instrument 128 to place securement elements 30 on the interior stomach wall 25. In this embodiment, the securement instrument 128 includes a suturing cartridge 130, but it could instead include a cartridge configured for stapling, gluing, welding, tissue joining, etc. Suturing cartridge 130 is attached to a distal track 135. In order to make the desired multiple attachments, suture cartridge 130 is either movable along a distal track 135 within the stomach, or multiple suturing cartridges are fixed at locations along distal track 135.

Referring still to FIGS. 11-19, there are shown sequential steps of a preferred embodiment, whereby the endoscopic instrument 128 provides track 135 on which suturing cartridge 130 follows in order to place suture stitches 140 into the stomach wall 25 in order to form plications 20 and to form stoma 15.

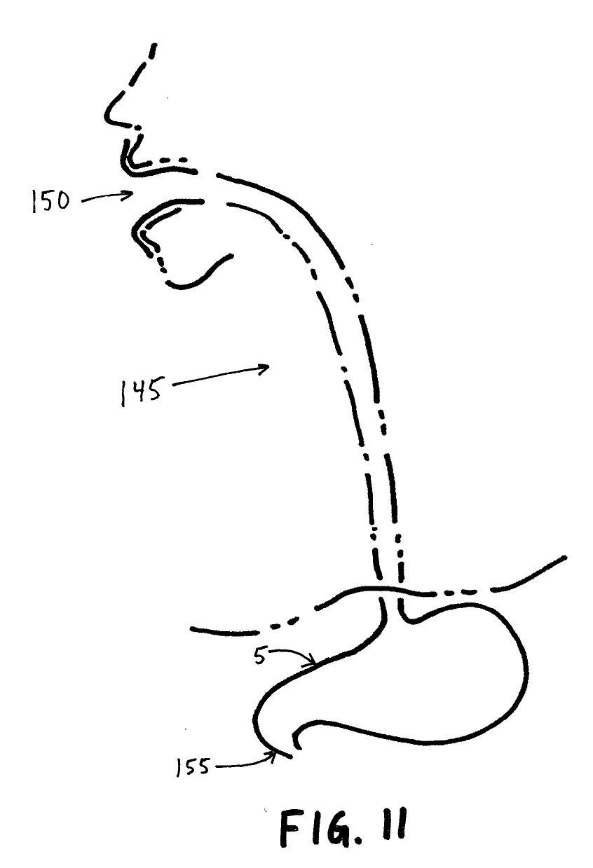

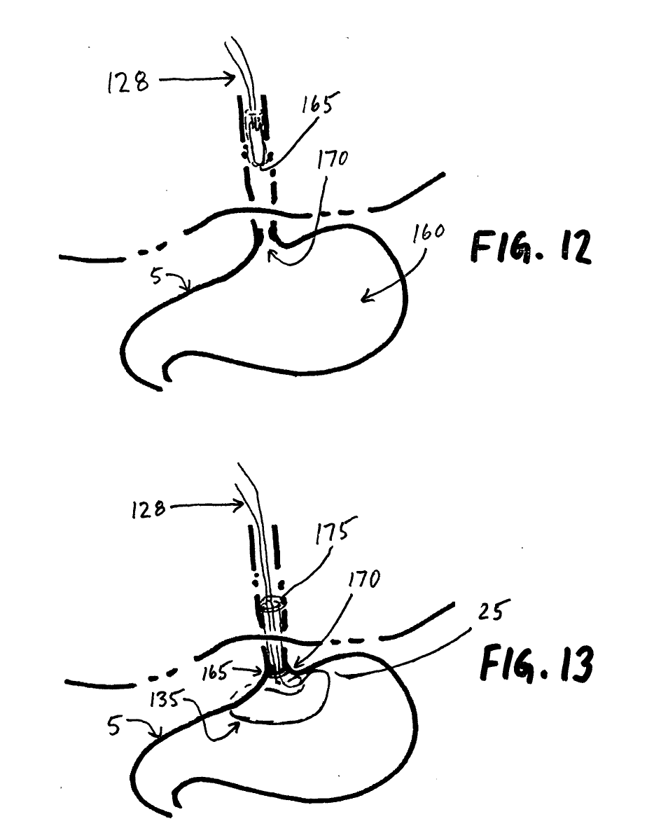

In FIG. 11, there is shown a side view of a human's upper gastrointestinal tract 145 from the mouth 150 leading to the pylorus 155. In FIG. 12, there is shown the instrument 128 being delivered endoscopically just before it reaches the interior portion 160 of stomach 5. In FIG. 13, there is shown distal end 165 of instrument 128 reaching interior portion 160 of stomach 5. Distal end 165 of instrument 128 is also expanded to form a circumferential loop 135 near the gastroesophageal junction 170 (GEJ) and in contact with the interior wall 25 of stomach 5. Instrument 128 also has an expandable element 175 that permits the instrument 128 to become removably affixed to the GEJ 170 such that distal loop 135 of the instrument is held in the desired position against interior wall 20 of stomach 5. Referring now to FIG. 14, there is shown suture cartridge 130 traveling down track 135A toward the distal loop 135 of instrument 128.

Referring to FIG. 15, there is shown suture cartridge 130 positioned on distal loop 135 such that a needle and suture (not shown) can be passed through the wall of stomach 5 to form stitches at a precisely desired first location 180.

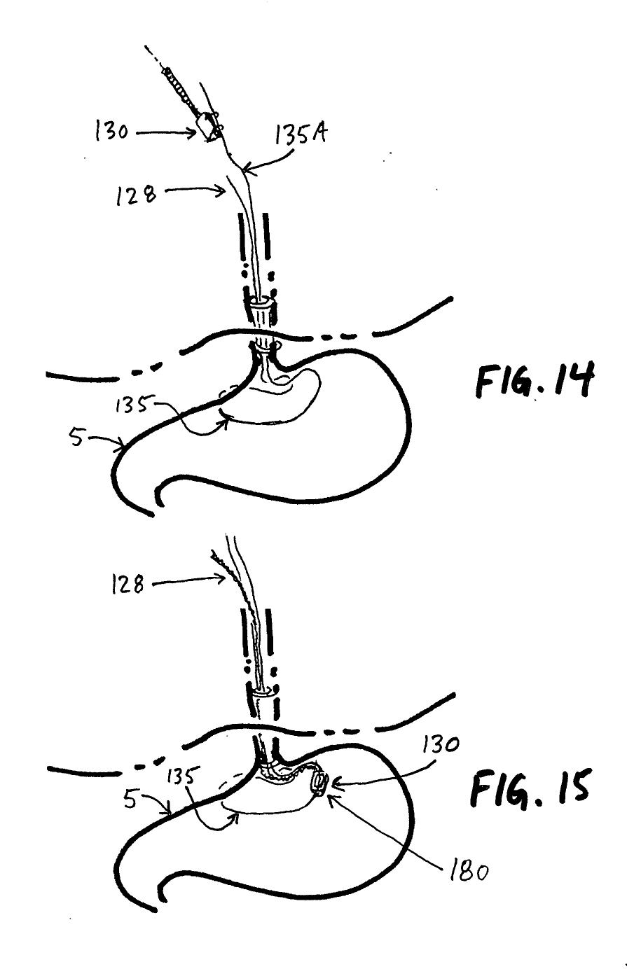

In FIG. 16, there is shown the suture cartridge 130, having been moved such that another needle and suture (not shown) can be passed through the wall 25 of stomach 5 to form stitches 140 at a precisely desired second location 185.

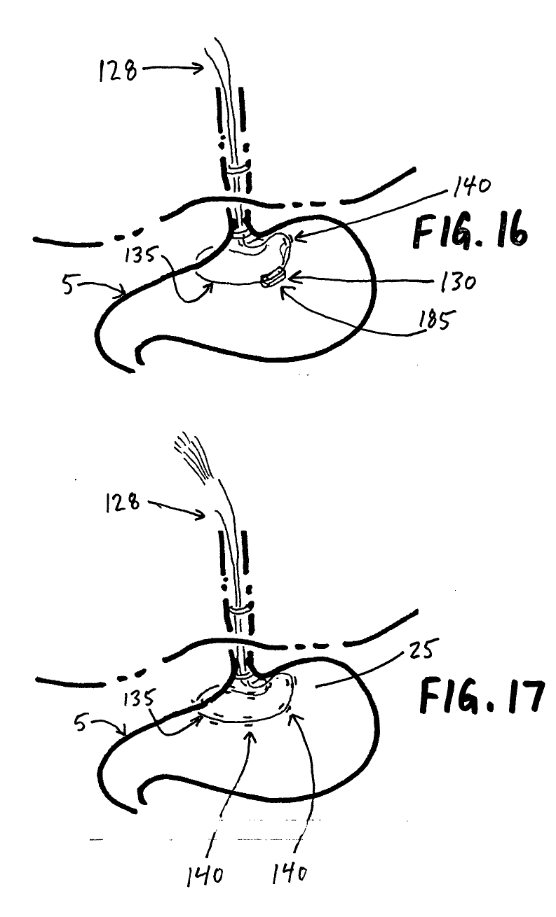

In FIG. 17, there is shown instrument 128 with suture cartridge 130 removed after having deployed the desired number of sutures 140 at specific locations in a nearly circumferential manner around the interior anterior wall 20 (posterior wall sutures not shown) of stomach 5.

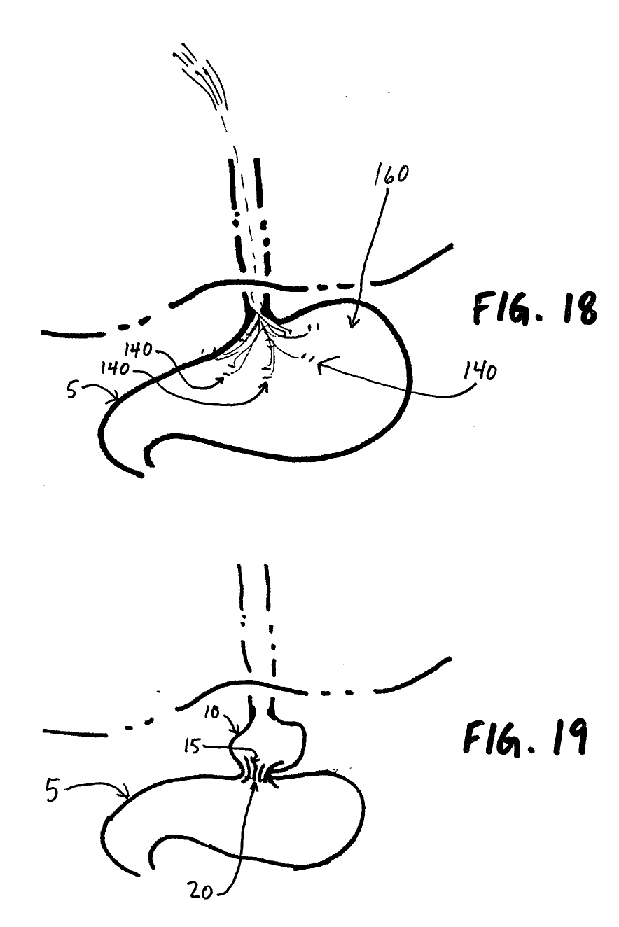

In FIG. 18, there is shown the withdrawal of instrument 128 from interior 160 of stomach 5, leaving sutures 140 that had been passed at specific locations.

In FIG. 19, there is shown a resultant stoma 15 created by tying knots on or otherwise crimping sutures 140. By tying knots on the sutures 140, plications 20 are formed and interconnected, collectively forming a stoma 15.

Referring now to FIGS. 20-24, there are shown plication techniques including endoscopic suturing to partition the stomach so as to create a small gastric pouch 10 (FIG. 1H) and a restrictive stoma 15 (FIG. 1G), whereby to provide a method to treat obesity in a less invasive manner than current surgical approaches. A preferred method to create a gastric pouch 10 (FIG. 1H) and stoma 15 (FIG. 1G) is to form a series of gastroplications as described herein.

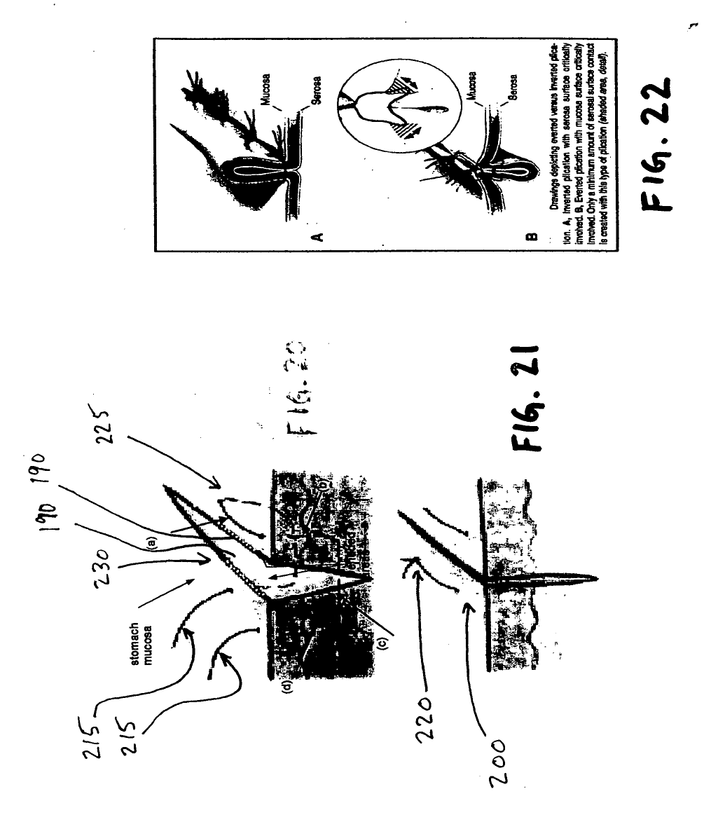

In accordance with the present invention as disclosed herein, a preferred embodiment of the present invention is designed to utilize the healing response of intraluminal gastric plications 20. Healing occurs between multiple joined folds 20. More particularly, the desired tissue joinder is achieved by effecting the plication 20, with full thickness suture penetration depth, and denuding of mucosa at areas 190 to gain seromuscular juxtaposition, and a fixation pattern 195 intended to maximize seromuscular juxtaposition. Successful intraluminal formation and maintenance of a healed plication 200 (FIG. 21) is a fundamental building block for endoscopically creating small gastric pouch 10 (FIG. 1H) and stoma 15 (FIG. 1G).

In order to create pouch 10 (FIG. 1H) and stoma 15 (FIG. 1G) in a minimally invasive manner, e.g., endoscopically, lasting gastroplications 20 should be created. Surgically created gastroplications 20 are one method of creating pouch 10 (FIG. 1H) and stoma 15 (FIG. 1G). More preferably, instrumentation and procedures are used to create lasting gastroplications endoscopically, thereby causing less pain and distress to the patient.

Referring now to FIGS. 20-24, there is shown, by means of a gastrotomy 205, plications 210 placed to simulate an endoscopic approach. Plications 210 are created with USP #2-0 non-absorbable suture, e.g., Prolene, formed by full thickness suture penetration depth. Controlled energy (e.g., bipolar electrocautery) or mechanical stripping is applied to denude the mucosa. A horizontal mattress stitch referred to as an “HMS” fixation pattern is preferably used to secure plication. The controlled energy source or mechanical stripping ablates or removes the mucosa between the HMS suture entry and exit points to expose the seromuscular tissue, which creates bleeding or exposes vascularized surfaces for apposition. Each plication is created using an HMS 195. Sutures are placed transluminally with needle holders, joining the targeted folds. A knot 220 is created with 5 half hitches to complete a plication. Care is preferably taken when tying knots 220 to ensure secure seromuscular apposition, but also to prevent necrosis. Preferably, the surface contact area 190 of seromuscular tissue is maximized through the configuration of HMS fixation 195 and denuded mucosa at area 190.

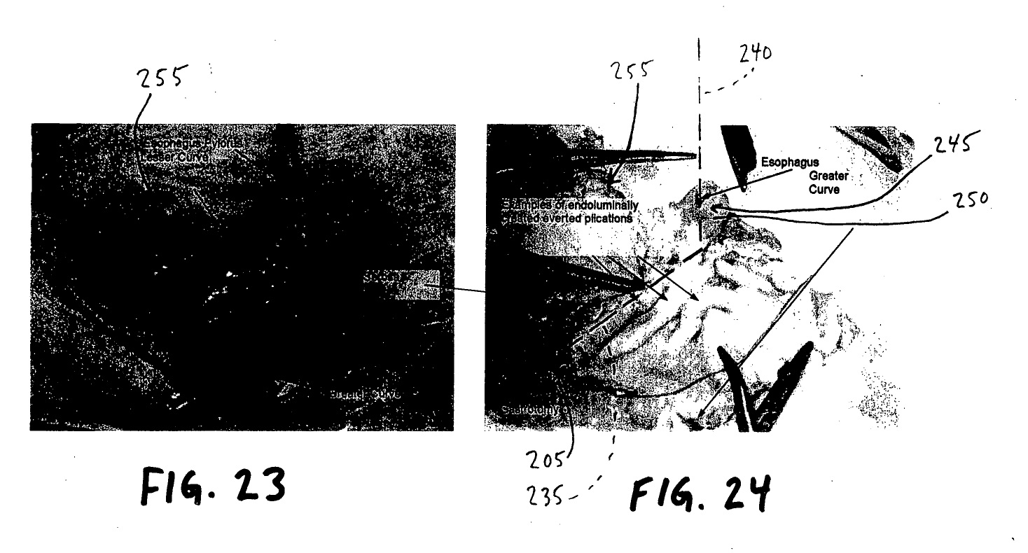

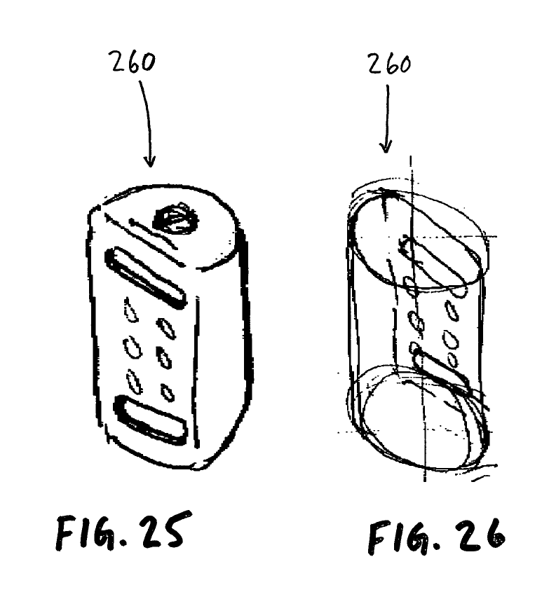

Referring to FIGS. 23 and 24, axis 235 of the HMS plications 210 are oriented toward the axis 240 of the esophagus 245 (see FIG. 24). A first endoluminal plication 210 is made on the anterior wall, approximately 5-6 cm from the gastroesophageal junction 250 (GEJ) and approximately 5-7 cm from the lesser curvature 255. Adjacent endoluminal plications are made approximately 2-3 cm from axis 235 of the previously created plication 210.

The gastrotomy 205 is closed with one layer of interrupted USP #2-0 non-absorbable sutures, the abdominal cavity rinsed with saline solution, and the abdominal wall closed.

In order to obtain a greater degree of tissue apposition, one or more of the following techniques are used including, but not limited to, more aggressive denuding of mucosa at area 190 (FIG. 20) to cause bleeding; different means of denuding, e.g., laser; increasing surface contact area, e.g., multiple mattress stitches, bolsters, more stitches to spread out load, etc.; and prescription of acid blockers.

Looking at FIGS. 25-31, and in accordance with the present invention as disclosed herein, there is shown a tissue attachment instrument 260 which can be used to create a stoma using suture material and a resultant tissue attachment pattern 265. There is shown a front view (FIG. 25) and rear view (FIG. 26) of the operating end of endoscopic tissue-to-tissue attachment instrument 260.

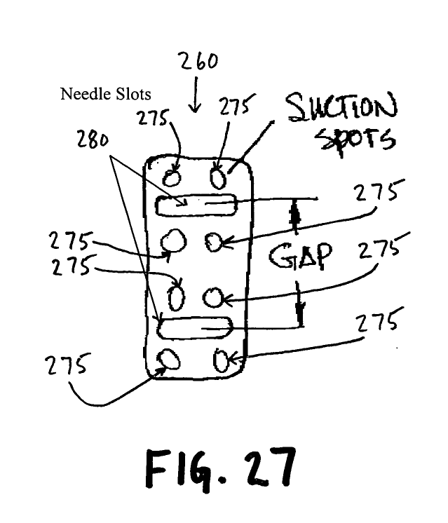

Looking at FIG. 27, there is shown a tissue contacting surface 270 of the operating end of tissue attachment instrument 260. Various apertures 275 are situated therein to provide control mechanisms, such as vacuum suction, referred to as suction spots, so as to control the tissue 25 (FIGS. 1C-1G). Parallel needle slots 280 are provided through which curved needles (not shown in FIG. 27) are passed through the controlled tissue 25 (FIGS. 1C-1G). Alternative control devices use grasping, hooking, suction or other forces to draw tissue into the operating end of contacting surface 270. The alternative control devices are configured to join tissue areas together using staples, clips, sutures, welding, etc.

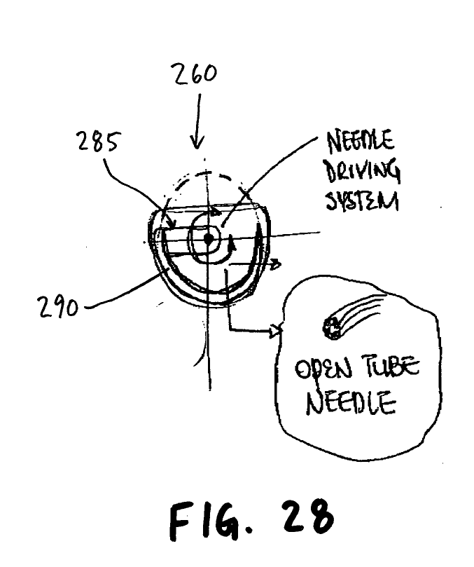

Looking at FIG. 28, there is shown a top cross-sectional view of instrument 260 having a control device 285 to drive a curved needle 290 through needle slot 280 and into tissue 25 (FIGS. 1C-1G).

Referring to FIG. 29, and in a preferred embodiment of the present invention, there is shown pattern 265 of a suture path a-b-c-d using a double-armed suture and driving the needles through the tissue 20.

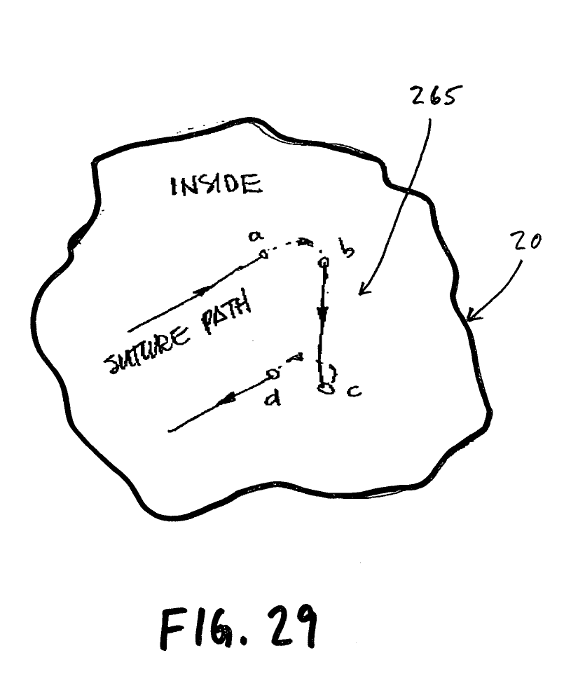

Looking at FIG. 30, there is shown tissue 20 brought into approximation by applying tension to suture 30 after being passed through a-b-c-d pattern 265 as shown in FIG. 29.

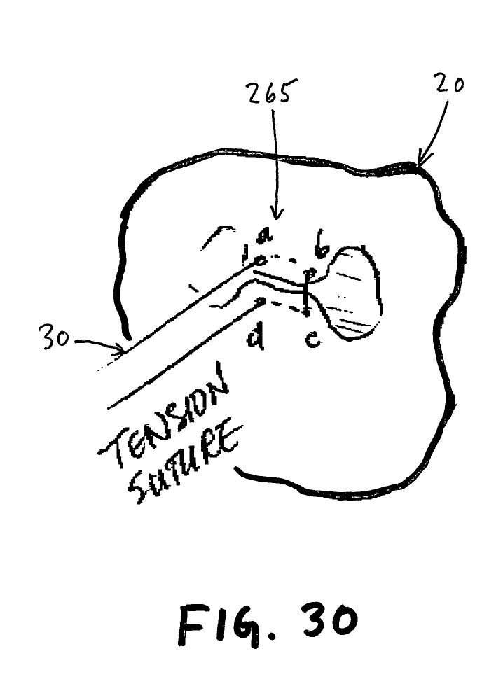

Referring now to FIG. 31, there is shown an end result with suture 30 pulled tightly, allowing the tissue 20 to heal between the suture lines a-b and c-d so as to provide long-term apposition. Prior to passing suture 30 through tissue 20, denuding the mucosa (or submucosa) to get bleeding and to expose the muscle layers will encourage healing and ultimately provide long-term strength to stoma 15 (FIG. 1G).

By approximating tissue 20 at various points circumferentially around GEJ 300 (FIG. 8), a stoma 15 (FIG. 1G) is created. The diameter of stoma 15 is adjusted in a number of ways. One preferred method to adjust the stoma diameter is to pass a suture through a′-b′-c′-d′ (FIG. 31) and cinching the suture 30, in effect doubling over the previously sutured a-b-c-d approximated tissue. Various other attachment elements 30, such as suture, staple, clip, glue, anchor, tissue welding, tissue approximation configurations and methods may be used in accordance with the present invention to create or adjust stoma 15 (FIG. 1G). In addition, a pouch sizing element in the form of inflatable balloon 120 (FIGS. 9 and 10) may be incorporated onto delivery tool 40 proximal to stoma 15 (FIG. 1G).

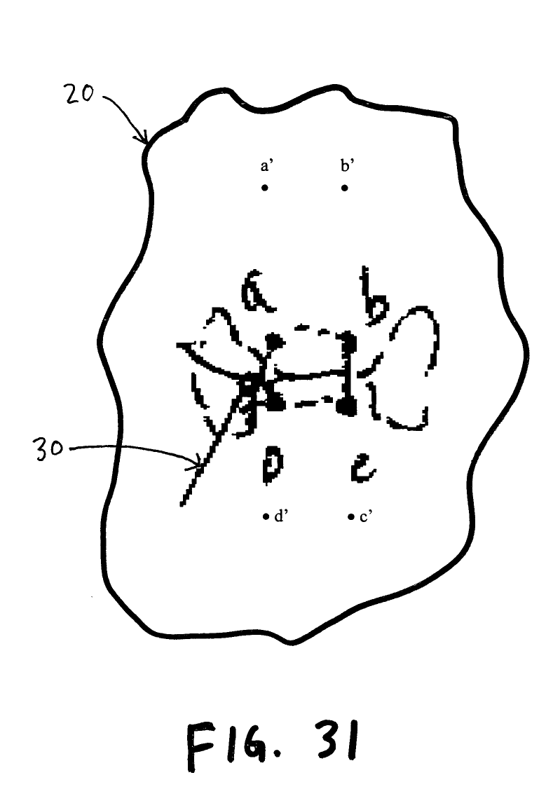

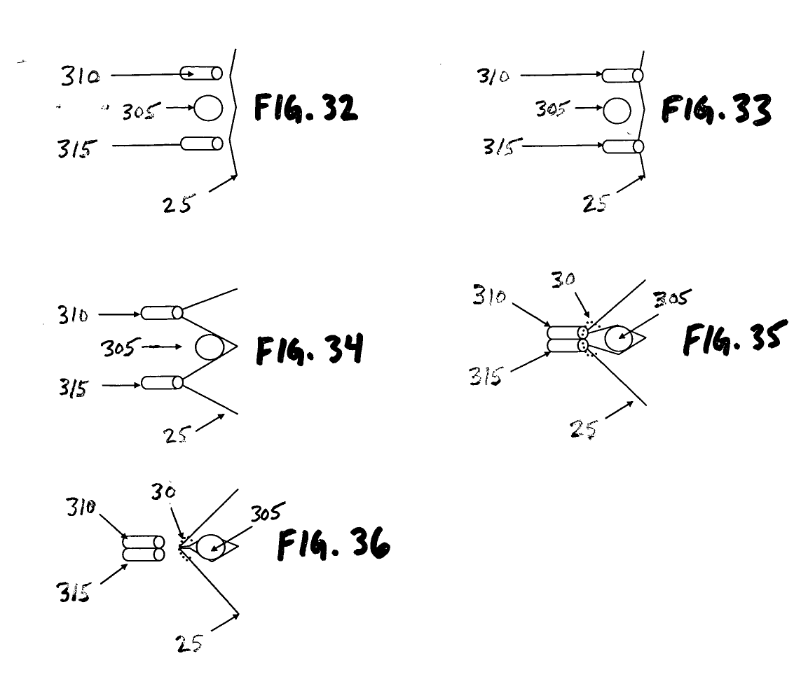

Referring now to FIGS. 32-36, there is shown a sequential endoscopic method for forming a stoma 15 by using a ring 305 or other device as a template for positioning the tissue 25 within the stomach. Looking at FIG. 32, there is shown a cross-section of ring 305 positioned between two instrument elements 310, 315 adjacent to tissue 25 of the interior wall of stomach. Referring to FIG. 33, there is shown the placement of the two instrument elements 310, 315 onto tissue 25 of the interior surface of the stomach. Instrument elements 310, 315 are used to control tissue portions 25 of the interior stomach wall and may be in the form of suction, grasping or other acceptable mechanisms. In FIG. 34, there is shown two instrument elements 310, 315 pulling parts of tissue 25 of the interior stomach wall such that ring 305 is embedded within tissue 25 of the stomach wall. In FIG. 35, there are shown two instrument elements 310, 315 being positioned adjacent to each other, drawing tissue 25 of the stomach wall completely around the ring 305. Also, a tissue attachment device (not shown) is introduced into the stomach wall at the appropriate place to secure tissue 20 of the stomach wall with attachment elements 30 such as to close the tissue over the ring 305. In FIG. 36, there is shown a release of tissue 25 of the stomach wall by instrument elements 310, 315 and the complete anchoring of the stomach wall around ring 305 using attachment elements 30 placed by the tissue attachment device. In this manner, stoma 15 (FIG. 1G) is created endoscopically to restrict the volume of stomach 5 or to delay the emptying of food from stomach 5.

It will be understood that many additional changes in the details, materials, steps and arrangements of parts, which have been herein described and illustrated in order to explain the nature of the invention, may be made by those skilled in the art within the principles and scope of the invention as expressed in the appended claims.

1. A method for restricting food volume capacity of the stomach, the method comprising:
manipulating tissue of an interior wall of the stomach from within the stomach so as to create an upper pouch, a lower pouch and an opening connecting the upper pouch and the lower pouch, the upper pouch being located adjacent the gastroesophageal junction, the lower pouch being located adjacent the pylorus, and the opening having a periphery formed by drawing together the tissue of the interior wall of the stomach.

2. A method according to claim 1 wherein the upper pouch is configured with a predetermined volume.

3. A method according to claim 1 wherein the opening is a stoma.

4. A method according to claim 1 wherein the tissue is drawn together by plication.

5. A method according to claim 1 wherein the opening has a given diameter formed by the periphery of drawn-together tissue.

6. A method for restricting food volume capacity of the stomach, the method comprising:
placing a plurality of elements for gripping the tissue within the stomach at given locations;
drawing the plurality of elements for gripping the tissue toward one another so as to draw the given locations of the stomach toward one another; and
fixing the position of the drawn-together locations of the stomach relative to one another;
whereby to divide the stomach into an upper pouch and a lower pouch, with an opening connecting the upper pouch to the lower pouch.

7. A method according to claim 6 wherein the step of fixing the position of the drawn-together locations of the stomach comprises suturing tissue at one of the given locations to tissue at another one of the given locations.

8. A method according to claim 6 wherein the step of fixing the position of the drawn-together locations of the stomach comprises gluing tissue at one of the given locations to tissue at another one of the given locations.

9. A method according to claim 6 wherein the step of fixing the position of the drawn-together locations of the stomach comprises stapling tissue at one of the given locations to tissue at another one of the given locations.

10. A method according to claim 6 wherein the step of fixing the position of the drawn-together locations of the stomach comprises tying adjacent ones of the plurality of suture to one another.

11. A method according to claim 6 further comprising, prior to the step of placing the plurality of elements for gripping the tissue, the step of debriding tissue so as to facilitate long-term tissue fixation.

12. A method for restricting food volume capacity of the stomach, the method comprising:
drawing toward one another given portions of tissue of an interior wall of the stomach from within the stomach; and
fixing the position of the given portions of the stomach drawn toward one another;
whereby to divide the stomach into an upper chamber and a lower chamber, with an opening connecting the upper chamber to the lower chamber.

13. A method according to claim 12 wherein the step of drawing toward one another given portions of tissue of the interior wall of the stomach from within the stomach comprises use of a device having arms configured to selectively position a plurality of elements for gripping the tissue to given locations on the tissue.

14. A method according to claim 13 wherein at least one of the arms of the device are configured so as to have a degree of extension different from the degree of extension of another one of the arms.

15. A method according to claim 13 wherein the arms of the device provide a vacuum to grasp and position the given portions of tissue.

16. A method according to claim 12 wherein the step of drawing toward one another the given portions of tissue of the interior wall of the stomach from within the stomach comprises use of a device having a track configured for positioning relative to the tissue and for guiding placement of a plurality of elements for gripping the tissue, whereby drawing the elements toward one another will cause the given portions of tissue to be drawn toward one another.

17. A method according to claim 12 wherein the step of drawing toward one another the given portions of tissue of the interior wall of the stomach from within the stomach is preceded by the use of a device having inflatable elements for inflation to stretch the walls within the stomach, whereby drawing the elements toward one another will cause the given portions of tissue to be drawn toward one another.

18. A device for creating a restriction in the stomach, the device comprising:
a retractor configured for deployment through the esophagus into the stomach;
a plurality of arms selectively deployable from the retractor, each of the plurality of arms configured to position a securement device adjacent to a given location of tissue of the stomach from within the stomach; and
a deployment mechanism for deploying the securement device positioned by each of the plurality of arms to the given location of the tissue of the stomach from within the stomach.

19. A device for creating a restriction in the stomach, the device comprising:
a track configured for deployment through the esophagus into the stomach;
a securement device mounted to the track, such that the securement device is positioned adjacent to a given location of tissue of the stomach from within the stomach; and
a deployment mechanism for deploying the securement device to the given location of tissue of the stomach from within the stomach.

20. A device according to claim 19 wherein the track comprises a superelastic material.

21. A device according to claim 19 wherein the securement device is slidably mounted to the track.
